(19) United States
(12) Patent Application Publication
(10) Pub. No.: US 2017/0269180 A1
(43) Pub. Date: Sep. 21, 2017

(54) SYSTEMS AND RELATED METHODS FOR RAPIDLY MOVING MATERIALS INTO AND OUT OF A CRYOGENIC ENVIRONMENT

(71) Applicant: MILLIKELVIN TECHNOLOGIES LLC, Braintree, MA (US)

(72) Inventors: Neal Kalechofsky, Stow, MA (US); Avrum BELZER, Brookline, MA (US)

(21) Appl. No.: 15/612,456

(22) Filed: Jun. 2, 2017

Related U.S. Application Data
(63) Continuation-in-part of application No. PCT/US2015/063169, filed on Dec. 1, 2015.
Application No. PCT/US2015/063169 is a continuation-in-part of application No. 15/230,739, filed on Aug. 8, 2016.
Application No. 15/230,739 is a continuation of application No. 14/190,945, filed on Feb. 26, 2014, now abandoned.
Application No. 14/190,945 is a continuation-in-part of application No. 13/844,446, filed on Mar. 15, 2013, now Pat. No. 9,207,298.
Application No. 13/844,446 is a continuation-in-part of application No. 13/623,759, filed on Sep. 20, 2012, now abandoned.
Application No. 13/623,759 is a continuation of application No. PCT/US2012/030384, filed on Mar. 23, 2012.
Application No. PCT/US2012/030384 is a continuation-in-part of application No. 15/230,739, filed on Aug. 8, 2016.
Application No. 15/230,739 is a continuation of application No. 14/190,945, filed on Feb. 26, 2014, now abandoned.
Application No. 14/190,945 is a continuation-in-part of application No. 13/844,446, filed on Mar. 15, 2013, now Pat. No. 9,207,298.
Application No. 13/844,446 is a continuation-in-part of application No. 15/230,739, filed on Aug. 8, 2016.
Application No. 15/230,739 is a continuation of application No. 14/190,945, filed on Feb. 26, 2014, now abandoned.
Application No. 14/190,945 is a continuation-in-part of application No. 13/335,076, filed on Dec. 22, 2011, now Pat. No. 8,703,201.
Application No. 13/335,076 is a continuation of application No. 12/193,536, filed on Aug. 18, 2008, now abandoned.
Application No. 12/193,536 is a continuation of application No. PCT/US2007/004654, filed on Feb. 21, 2007.
Application No. PCT/US2007/004654 is a continuation-in-part of application No. 15/230,739, filed on Aug. 8, 2016.
Application No. 15/230,739 is a continuation of application No. 14/190,945, filed on Feb. 26, 2014, now abandoned.
Application No. 14/190,945 is a continuation-in-part of application No. 12/879,634, filed on Sep. 10, 2010, now Pat. No. 8,703,102.
Application No. 12/879,634 is a continuation of application No. PCT/US10/47310, filed on Aug. 31, 2010.
Application No. PCT/US10/47310 is a continuation-in-part of application No. 15/230,739, filed on Aug. 8, 2016.
Application No. 15/230,739 is a continuation of application No. 14/190,945, filed on Feb. 26, 2014, now abandoned.
Application No. 14/190,945 is a continuation-in-part of application No. 12/879,634, filed on Sep. 10, 2010, now Pat. No. 8,703,102.
Application No. 12/879,634 is a continuation-in-part of application No. PCT/US2009/039696, filed on Apr. 6, 2009.
(60) Provisional application No. 62/086,475, filed on Dec. 2, 2014.
(60) Provisional application No. 61/466,500, filed on Mar. 23, 2011.
(60) Provisional application No. 61/522,076, filed on Aug. 10, 2011.
(60) Provisional application No. 61/667,283, filed on Jul. 2, 2012.
(60) Provisional application No. 61/706,100, filed on Sep. 26, 2012.
(60) Provisional application No. 61/706,102, filed on Sep. 26, 2012.
(60) Provisional application No. 61/706,106, filed on Sep. 26, 2012.
(60) Provisional application No. 61/733,415, filed on Dec. 4, 2012.
(60) Provisional application No. 60/775,196, filed on Feb. 21, 2006.
(60) Provisional application No. 60/802,699, filed on May 23, 2006.
(60) Provisional application No. 61/238,647, filed on Aug. 31, 2009.
(60) Provisional application No. 61/042,398, filed on Apr. 4, 2008.
(60) Provisional application No. 61/111,050, filed on Nov. 4, 2008.

(51) Int. Cl. G01R 33/56; A61B 5/00; A61B 5/026; G01R 33/36; G01R 33/465; G01R 33/48; G01R 33/563; G01R 33/46

(52) U.S. Cl. CPC G01R 33/5601; A61B 5/0263; G01R 33/56308; G01R 33/56366; G01R 33/465; G01R 33/48; A61B 5/748; G01R 33/3607; A61M 2205/057; G01R 33/46; G01R 33/36; A61B 2576/00

(57) ABSTRACT

Disclosed herein is a device defining a generally closed volume therein, henceforth known as a “shuttle”, not permanently fixed to a probe or other surface inside the cryostat, into which gas and/or liquid—most preferably helium gas or liquid—can pass into or out of in a controlled and predictable manner. The passage of gas or liquid into the shuttle is preferably via a porous barrier so that sterile conditions can be maintained in the interior of the shuttle.

### CROSS-REFERENCE TO RELATED APPLICATIONS

This patent application is a continuation-in-part of International Application No. PCT/US2015/063169, filed Dec. 1, 2015, which in turn claims the benefit of priority to U.S. Provisional Application Ser. No. 62/086,475, filed Dec. 2, 2014.

This application claims the benefit of priority of and is a continuation-in-part of U.S. patent application Ser. No. 15/230,739, filed Aug. 8, 2016, which in turn claims the benefit of priority of and is a continuation of U.S. patent application Ser. No. 14/190,945, filed Feb. 26, 2014; which in turn is a continuation-in-part of U.S. patent application Ser. No. 13/844,446, filed Mar. 15, 2013, now U.S. Pat. No. 9,207,298, issued Dec. 8, 2015; which in turn is a continuation-in-part of U.S. patent application Ser. No. 13/623,759, filed Sep. 20, 2012; which in turn claims the benefit of priority to and is a continuation of International Patent Application No. PCT/US2012/030384, filed Mar. 23, 2012; which in turn claims the benefit of priority to U.S. Provisional Patent Application Ser. No. 61/466,500, filed Mar. 23, 2011; and U.S. Provisional Patent Application Ser. No. 61/522,076, filed Aug. 10, 2011.

This application claims the benefit of priority of and is a continuation-in-part of U.S. patent application Ser. No. 15/230,739, filed Aug. 8, 2016, which in turn claims the benefit of priority of and is a continuation of U.S. patent application Ser. No. 14/190,945, filed Feb. 26, 2014; which in turn is a continuation-in-part of U.S. patent application Ser. No. 13/844,446, filed Mar. 15, 2013, now U.S. Pat. No. 9,207,298, issued Dec. 8, 2015; which in turn claims the benefit of priority to U.S. Provisional Patent Application Ser. No. 61/667,283, filed Jul. 2, 2012; U.S. Provisional Patent Application Ser. No. 61/706,100, filed Sep. 26, 2012; U.S. Provisional Patent Application Ser. No. 61/706,102, filed Sep. 26, 2012; U.S. Provisional Patent Application Ser. No. 61/706,106, filed Sep. 26, 2012; and U.S. Provisional Patent Application Ser. No. 61/733,415, filed Dec. 4, 2012.

This application claims the benefit of priority of and is a continuation-in-part of U.S. patent application Ser. No. 15/230,739, filed Aug. 8, 2016, which in turn claims the benefit of priority to and is a continuation of U.S. patent application Ser. No. 14/190,945, filed Feb. 26, 2014, which in turn is a continuation-in-part of U.S. patent application Ser. No. 13/335,076, filed Dec. 22, 2011, now U.S. Pat. No. 8,703,201, issued Apr. 22, 2014; which in turn claims the benefit of priority from and is a continuation of U.S. patent application Ser. No. 12/193,536, filed Aug. 18, 2008; which in turn claims the benefit of priority to and is a continuation of International Application No. PCT/US2007/004654, filed Feb. 21, 2007; which in turn claims the benefit of priority of U.S. Provisional Patent Application Ser. No. 60/775,196, filed Feb. 21, 2006; and U.S. Provisional Patent Application Ser. No. 60/802,699 filed May 23, 2006.

This application claims the benefit of priority of and is a continuation-in-part of U.S. patent application Ser. No. 15/230,739, filed Aug. 8, 2016, which in turn claims the benefit of priority to and is a continuation of U.S. patent application Ser. No. 14/190,945, filed Feb. 26, 2014; which in turn is a continuation-in-part of U.S. patent application Ser. No. 12/879,634, filed Sep. 10, 2010, now U.S. Pat. No. 8,703,102, issued Apr. 22, 2014; which in turn is a continuation of and claims the benefit of priority of International Application No. PCT/US2010/047310, filed Aug. 31, 2010; and which in turn claims the benefit of priority to U.S. Provisional Patent Application Ser. No. 61/238,647, filed Aug. 31, 2009.

This application claims the benefit of priority of and is a continuation-in-part of U.S. patent application Ser. No. 15/230,739, filed Aug. 8, 2016, which in turn claims the benefit of priority to and is a continuation of U.S. patent application Ser. No. 14/190,945, filed Feb. 26, 2014; which in turn is a continuation-in-part of U.S. patent application Ser. No. 12/879,634, filed Sep. 10, 2010, now U.S. Pat. No. 8,703,102, issued Apr. 22, 2014; which in turn is a continuation in part of and claims the benefit of priority of International Application No. PCT/US2009/039696, filed Apr. 6, 2009; which in turn claims the benefit of priority of U.S. Provisional Patent Application Ser. No. 61/042,398, filed Apr. 4, 2008; and U.S. Provisional Patent Application Ser. No. 61/111,050, filed Nov. 4, 2008.

The disclosure of each of the aforementioned patent applications is incorporated by reference herein in its entirety for any purpose whatsoever.

### BACKGROUND

For many applications, both research and industrial, it is desirable to expose materials to a cryogenic environment. Cold temperatures can be used to induce changes of state (such as the transition from a liquid to a solid), cause materials to undergo a desired amount of stress, cause large variations in material characteristics such as its electrical conductivity, and so on. This list is representative and is not intended to be exhaustive. The present disclosure provides improvements to the state of the art as described below.

### SUMMARY

Often the most time consuming portion of exposing a material to cryogenic temperatures involves cooling it to the desired temperature. Applicant has observed that, if a gas or liquid is not used for thermal contact, then the material must be somehow mechanically grounded to a source of cold. If a liquid or gas is used, such as liquid nitrogen or liquid helium, the material must generally be secured to some kind of probe or “stick” so that it can be removed from the low temperature environment when desired.

Applicant has found this approach to be undesirable for at least two reasons. First, the probe itself conducts heat into the low temperature environment (“LTE”). Second, removal of the stick is generally slow and cumbersome. Such sticks are often more than a meter long and made from stainless steel.

Applicant has also observed that there are also applications where it is not possible to attach the material to be cooled to any kind of probe or stick. For example, it is possible to hyperpolarize (HP) a material for use in a medical imaging study by cooling it to temperatures less than 4 K in a high field magnet. In order to retrieve it from the cryostat without loss of polarization it must be removed rapidly. In this case sample ejection time on the order of one second is necessary which would not be practical if the target material were attached to a stick.

One solution is to contain the material in a volume which can be expelled from the LTE using gas pressure. This approach has been used, for example, in Helium-Deuterium (HD) fusion experiments where the HD pellet is accelerated to high velocity inside a “sabot” which later breaks away allowing the pellet to fly towards the Tokamak. This approach, however, only works for materials like HD that can be frozen in situ into a pellet. Also, the approach is not amenable for materials that must be kept sterile.

U.S. patent application Ser. No. 14/212,695, filed Mar. 14, 2014 (incorporated by reference herein in its entirety for any purpose whatsoever), (Published as US2014/0263359) describes a volume that was used in HP experiments by the inventor of the present application. In that case the volume consisted of a cylinder open on both ends with a target material frozen in an annulus to the interior of the shuttle. That approach demonstrably yielded rapid extraction of sample from the polarizing cryostat.

However, Applicant has come to appreciate that this approach has its shortcomings. Applicant has noted that it was difficult to maintain low material temperatures during ejection as the shuttle passed through regions of warm gas outside the polarizing cryostat, and not practical as the desired reduction in heat transfer could not be accomplished without cooling the entire path that the shuttle traversed to an extremely low temperature. In addition, Applicant notes that the open geometry made it difficult to maintain sterility of the material since a low temperature environment, in and of itself, can not be assumed to produce sterile conditions. Finally, Applicant observed that this approach was only practical for transporting materials that are liquid at room temperature.

Applicant has come to appreciate that it would thus be advantageous to encase the polarized material completely within a volume. This can help keep the material sterile and help to maintain sample temperature during expulsion. However, this approach would at first blush be unsuitable since it is not possible to get materials very cold in this manner. Thermal contact through the walls of the volume, especially at T<4 K where thermal conductivity of most solids is very low, is generally very poor. In addition there is the possibility of any helium gas outside the volume eventually leaking in as many solids are porous to helium on some level over a sufficiently long time period. The liquid helium trapped in the volume would expand rapidly when the volume is ejected and warmed, and this could in turn cause an unsafe amount of pressure inside the volume.

### BRIEF DESCRIPTION OF THE FIGURES

FIG. 1 shows a photo of an example shuttle in accordance with the disclosure.

FIG. 2 is a schematic representation of a shuttle as shown in FIG. 1, containing a sample and cooled to T<1 K.

### DETAILED DESCRIPTION

Disclosed herein to address the aforementioned deficiencies in the art is a device defining a generally closed volume therein, henceforth known as a “shuttle”, not permanently fixed to a probe or other surface inside the cryostat, into which gas and/or liquid—most preferably helium gas or liquid—can pass into or out of in a controlled and predictable manner. The passage of gas or liquid into the shuttle is preferably via a porous barrier so that sterile conditions can be maintained in the interior of the shuttle at all times. Ideally the barrier is porous enough to permit easy passage of liquid or gas but can also prevent bacteria, microbes or other undesirable materials from entering the shuttle. In the case where the contents of the shuttle are to be used in a medical application, this will allow sterility to be maintained while the shuttle and its contents are either being cooled in the cryostat or ejected from it.

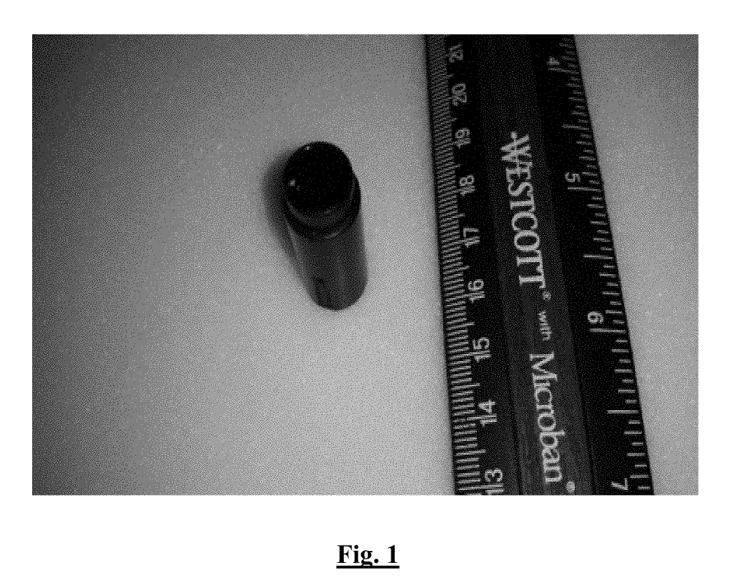

To accomplish this Applicant describes herein a closed shuttle wherein at least some portion of the surface of the shuttle is made from a porous material. In a preferred embodiment the material is Porex® material (from Porex Corporation, Fairburn, Ga., USA) or some other kind of porous plastic material wherein the pore size is engineered to have a desired average diameter. For example, plastic materials such as Ultra-high molecular weight polyethylene (UHMWPE), high-density polyethylene (HDPE), polypropylene (PP), polytetrafluoroethylene (PTFE), and polyvinylidene fluoride (PVDF), Ethylene vinyl acetate (EVA), polyethersulfone (PES), polyurethane (PU) and PE/PP co-polymers can be used. The Porex® material is thus designed to serve as a sterile barrier. FIG. 1 shows a photo of an example shuttle.

The shuttle can be made from a wide variety of materials, depending on its desired thermal characteristics. In a preferred embodiment, the shuttle is machined from a thermally conducting plastic that is magnetically inert. Examples of such materials can be found in the literature.

In some embodiments, the porous material defines pores therein having an average diameter between about 800 nm and about 50 nm, or any increment therebetween of about 10 nm. In another embodiment, the porous material defines pores therein having an average diameter between about 300 nm and about 100 nm, or any increment therebetween of about 10 nm. In some embodiments the porous material defines pores therein having an average diameter between about 250 nm and about 200 nm, or any increment therebetween of about 5 nm, such as 220 nm.

In a further preferred embodiment, the gas or liquid passing through the porous barrier is helium. Helium has two main isotopes, ⁴He and ³He. For purposes of this teaching the word “helium” is meant to describe pure ⁴He, pure ³He, or some combination of the two. Note liquid 3 He and liquid 4 He are miscible below approximately 867 mK.

In a further preferred embodiment, the material inside the shuttle is arranged to have a high surface area. High surface areas facilitate thermal relaxation, particularly in ultra low temperature environments where Kapitza resistance can be significant. Kapitza resistance arises due to “phonon mismatch” between a given material and liquid helium and can result in greatly increased thermal relaxation times. As is well described in the literature, the easiest route to overcoming Kapitza resistance is to configure the material in a high surface area format.

In a further preferred embodiment, the material inside the shuttle is suitable for use in a hyperpolarized NMR/MRI/MRS study. In a further preferred embodiment, the material in the shuttle is liquid 1-13 C pyruvic acid, where 1-13 C refers to the pyruvic acid being isotopically enhanced in the carbonyl atom position.

In a further preferred embodiment, the shuttle is exposed to a combination of very low temperatures and high magnetic fields suitable for producing high nuclear polarization in the material contained in it. Since after polarization the shuttle will be expelled from the LTE as rapidly as possible, it is desirable to make it out of a magnetically inert material. Various plastics or metals such as copper, brass etc are suitable for this.

Using such a shuttle, samples can be exposed to extremely low temperatures, well below T˜4 K, and still achieve rapid cooling if helium gas is used to provide thermal contact. Methods of producing temperatures down to 1 millikelvin or below are well known in the literature. In a preferred embodiment, a dilution refrigerator (DR) is used to produce temperatures down to T<10 mK with cooling powers ˜1 microwatt or more.

In U.S. Pat. No. 6,651,459, Applicant has separately taught a proprietary method of using ³He to enhance surface relaxation rates of a powder in a high B/T environment. In this method, ³He is allowed to condense onto the surface of the frozen powder. At temperatures below 3 K, and down to absolute zero, ³He is a liquid at normal pressures and will wet virtually any surface with which it comes into contact. Experiments on a wide range of nuclei and molecules have shown that as little as one monolayer of ³He on the surface of a material will cause nearby nuclei to relax to equilibrium at a highly enhanced rate. This phenomenon occurs because ³He atoms in the monolayer are continually exchanging sites with one another, even at temperatures approaching absolute zero. Dipolar coupling between the spin ½ ³He atoms and nuclei in the surface layers cause rapid relaxation of those nuclei to equilibrium polarization; the remainder of the sample relaxes via spin diffusion. In a preferred embodiment, ³He is allowed to pass through the Porex barrier and provide both enhanced thermal and magnetic relaxation to equilibrium.

The shuttle containing the material to be cooled is introduced into the LTE produced by a DR. In a preferred embodiment, this is done by allowing the shuttle to be let down an evacuated tube running between room temperature and the cold stage of the DR. The DR is also used to cool a prearranged amount of helium to a desired temperature. Once the shuttle is in the cold stage of the DR, helium is allowed into the tube and passes through the porous barrier in the shuttle. This provides thermal contact between the contents in the interior of the shuttle and the cold stage of the DR.

As is well described in the literature, helium can be used to provide good thermal contact between a material and the cold stage of a DR. This is because under its own vapour pressure helium remains a liquid even down to absolute zero. Also, the molar heat capacity of helium at T<4 K generally greatly exceeds the molar heat capacity of any other material. This makes it an excellent refrigerant for cooling materials to ultra low temperatures providing the material can be configured in a high surface area format to overcome Kapitza resistance.

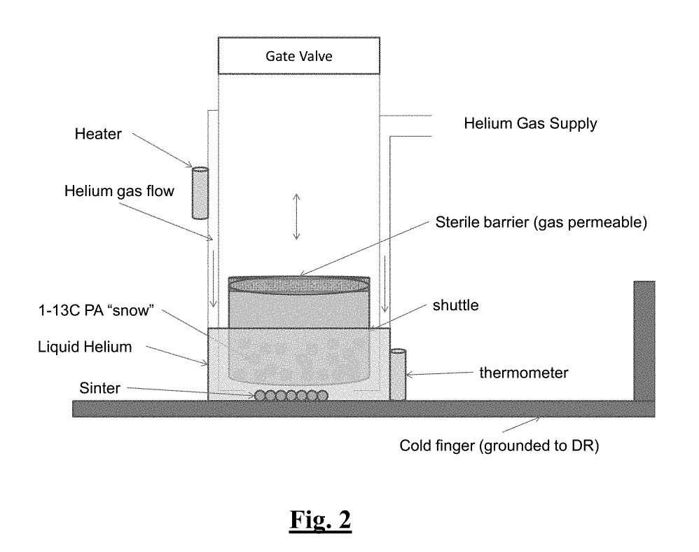

FIG. 2 shows an illustration of such a shuttle where it, and the sample it contains, (in a preferred embodiment, isotopically enhanced 1-13 C pyruvic acid) are cooled to T<1 K in a LTE created by a dilution refrigerator (DR). The shuttle is introduced into the LTE by dropping down a tube filled with helium gas. When it is desired to remove the sample from the LTE gas pressure can be used to accelerate it out of the cryostat. As helium gas or liquid within the shuttle warms it passes out of the Porex® barrier in a controlled fashion. This not only deters unwanted buildup of pressure, but the gas can also be expected to produce some cooling via the Joule-Thomson effect as it passes through the pores in the barrier. This will help to keep the sample inside the shuttle cold.

Samples can be loaded into the shuttle in a number of ways. If the sample includes materials that are gases or liquids at room temperature, they may be flowed through the barrier. The Porex® barrier is removable and resealable using epoxy, so solids can be loaded into the interior and then the Porex® (or other suitable) barrier can be sealed across the opening.

In a preferred embodiment, the shuttle is cylindrical in shape, but it can have any desired three-dimensional shape. The body of the shuttle can be made from a wide variety of materials. Also, if desired, a window can be fitted into the body of the shuttle—for example, if laser excitation of the sample within is desired. Windows can be made from quartz, aluminum, beryllium or other materials and secured to the body of the shuttle using well understood cryogenic engineering techniques.

Various hyperpolarization techniques can be used to hyperpolarize the material within the shuttle, including brute force hyperpolarization and hyperpolarization using a quantum relaxation switch, as described in detail in patents and patent applications incorporated by reference herein.

The methods, systems, and devices of the present disclosure, as described above and shown in the drawings, among other things, provide for improved magnetic resonance methods, systems and machine readable programs for carrying out the same. It will be apparent to those skilled in the art that various modifications and variations can be made in the devices, methods, and devices of the present disclosure without departing from the spirit or scope of the disclosure. Thus, it is intended that the present disclosure include modifications and variations that are within the scope of the subject disclosure and equivalents.

1. A device for rapidly moving materials into or out from a cryogenic environment, said device including a peripheral wall defining an interior volume made from a solid material including a porous portion made from a different material, the porous portion being porous to liquid or gas.

2. The device of claim 1, wherein the interior volume is cylindrical.

3. The device of claim 1, wherein the porous portion is porous to helium gas.

4. The device of claim 1, wherein the peripheral wall is made from high thermal conductivity plastic.

5. The device of claim 1, wherein the peripheral wall is made from low thermal conductivity plastic.

6. The device of claim 1, wherein the peripheral wall is made from PTFE.

7. The device of claim 1, wherein the porous portion is made from Porex® material.

8. The device of claim 1, wherein the interior volume maintains its sterility when passing through or into a non-sterile environment.

9. A method of conducting a NMR/MRI/MRS study using material hyperpolarized using the device of claim 1.

10. The device of claim 1, further comprising hyperpolarized material disposed within the interior volume of the device.

11. The device of claim 10, wherein the hyperpolarized material includes 1-13 C pyruvic acid.

12. The device of claim 10, wherein the hyperpolarized material includes frozen 1-13 C pyruvic acid disposed in a high surface area format.

13. The device of claim 1, wherein an exterior portion of the device permits the transmission of visible light therethrough.

14. The method of claim 9, wherein material inside the device is cooled to T<100 K during the method.

15. The method of claim 9, wherein material inside the device is cooled to T<10 K during the method.

16. The method of claim 9, wherein material inside the device is cooled to T<1 K during the method.

17. The method of claim 9, wherein the device is expelled from a cryogenic environment at a speed>0.1 m/s.

18. The method of claim 9, wherein the device is expelled from a cryogenic environment at a speed>1.0 m/s.

19. The method of claim 9, wherein the device is expelled from a cryogenic environment at a speed>10.0 m/s.
